(19) United States
(12) Patent Application Publication
(10) Pub. No.: US 2012/0220777 A1
(43) Pub. Date: Aug. 30, 2012

(54) PROCESS FOR THE PREPARATION OF A CRYSTALLINE FORM OF LENALIDOMIDE

(75) Inventors: Saridi Madhava Dileep Kumar, Gurgaon (IN); Munish Kapoor, Amritsar (IN); Swargam Sathyanarayana, Karim Nagar (IN); Rajesh Kumar Thaper, Jammu (IN); Mohan Prasad, Gurgaon (IN)

(73) Assignee: RANBAXY LABORATORIES LIMITED, New Delhi, Delhi (IN)

(21) Appl. No.: 13/395,922

(22) PCT Filed: Sep. 16, 2010
(86) PCT No.: PCT/IB2010/054187

§ 371 Date: May 1, 2012

(30) Foreign Application Priority Data
Sep. 16, 2009 (IN) 1930/DEL/2009

(51) Int. Cl. C07D 401/04

(52) U.S. Cl. 546/200

(57) ABSTRACT

The present invention relates to an in-situ process for the preparation of polymorphic Form A of lenalidomide.

### FIELD OF THE INVENTION

The present invention relates to a process for the preparation of polymorphic Form A of lenalidomide which involves in-situ crystallization.

### BACKGROUND OF THE INVENTION

Lenalidomide is an immunomodulatory agent with antiangiogenic and antineoplastic properties. Lenalidomide is indicated for the treatment of myelodysplastic syndromes and for the treatment of multiple myeloma. Lenalidomide is chemically 3-(4- amino-1-oxo-1,3-dihydro-2H-isoindol -2-yl)piperidine-2,6-dione of Formula I.

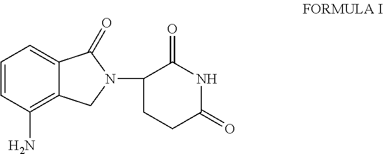

WO 2005/023192 describes polymorphic Forms A, B, C, D, E, F, G and H of lenalidomide. WO 2005/023192 says that Form A can be obtained from various solvents including 1-butanol, butyl acetate, ethanol, ethyl acetate, methanol, methyl ethyl ketone and tetrahydrofuran and provides XRPD, TGA, DSC, IR and Raman data on Form A.

Form A is described to be an unsolvated. However, there is no specific method for the preparation of Form A provided in available literature, including WO 2005/023192, which simply mentions that Form A can be prepared by recrystallization from several solvents.

### SUMMARY OF THE INVENTION

The present inventors have developed an in-situ crystallization process to obtain polymorphic Form A of lenalidomide without the need for isolating crude lenalidomide. The present process is simple and economical and it consistently provides polymorphic Form A of lenalidomide.

### BRIEF DESCRIPTION OF THE DRAWINGS

FIG. 1 depicts the X-Ray Powder Diffractogram (XRPD) of Form A of lenalidomide.

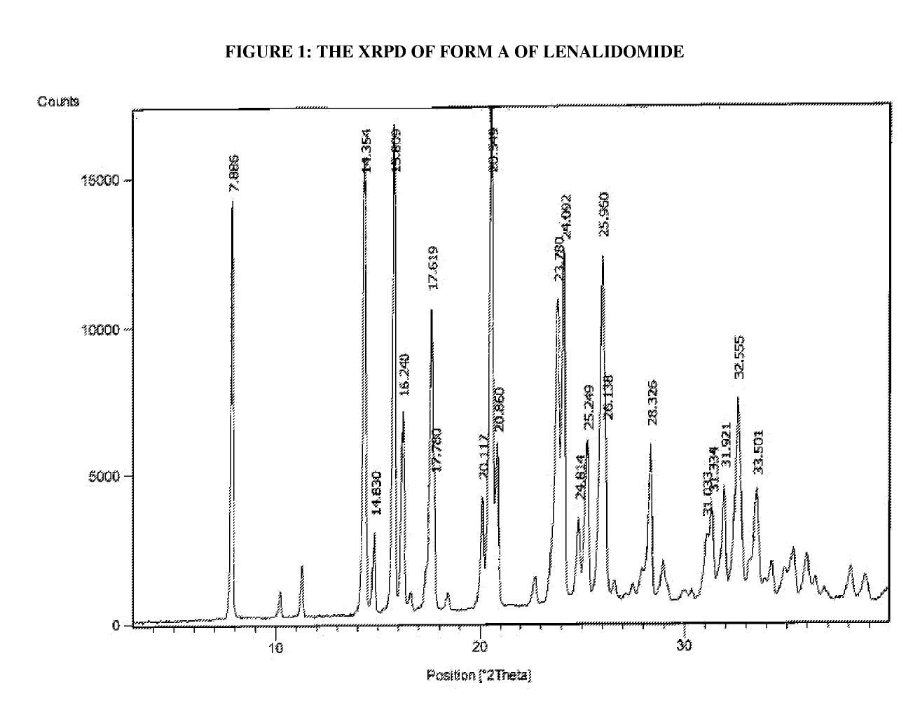

FIG. 1A provides the table of values for the XRPD of FIG. 1.

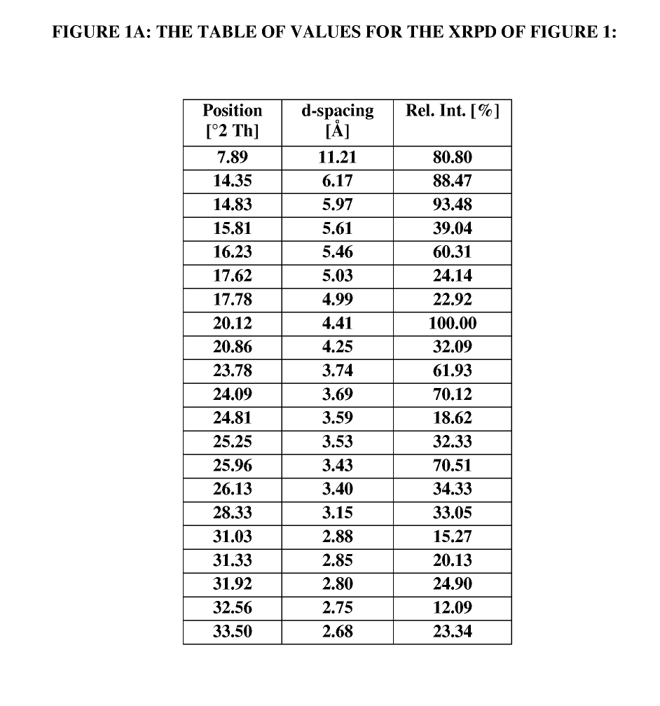

FIG. 2 depicts the Thermogravimetric Analysis (TGA) of Form A of lenalidomide.

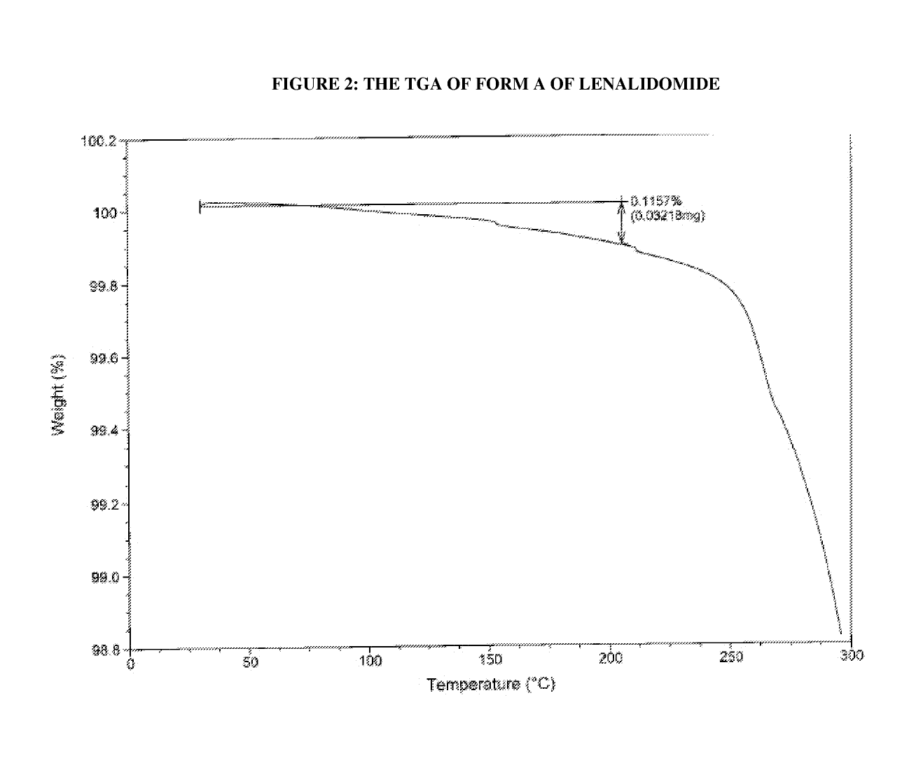

FIG. 3 depicts the Differential Scanning calorimetry (DSC) thermogram of Form A of lenalidomide.

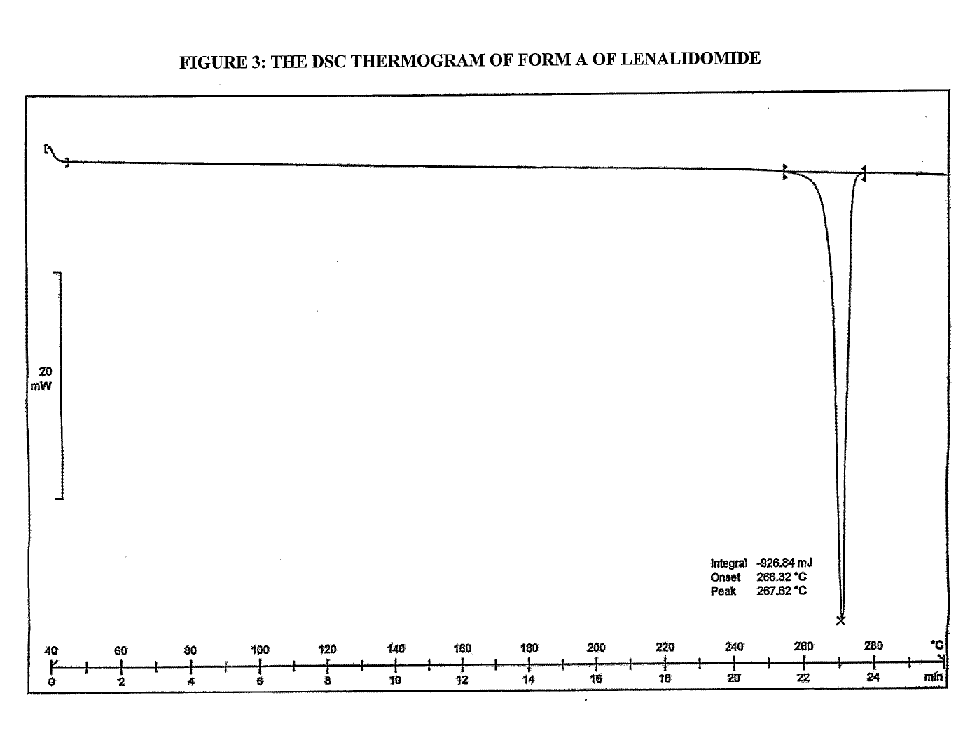

FIG. 4 depicts the Fourier-Transform Infra-red (FTIR) spectrum of Form A of lenalidomide.

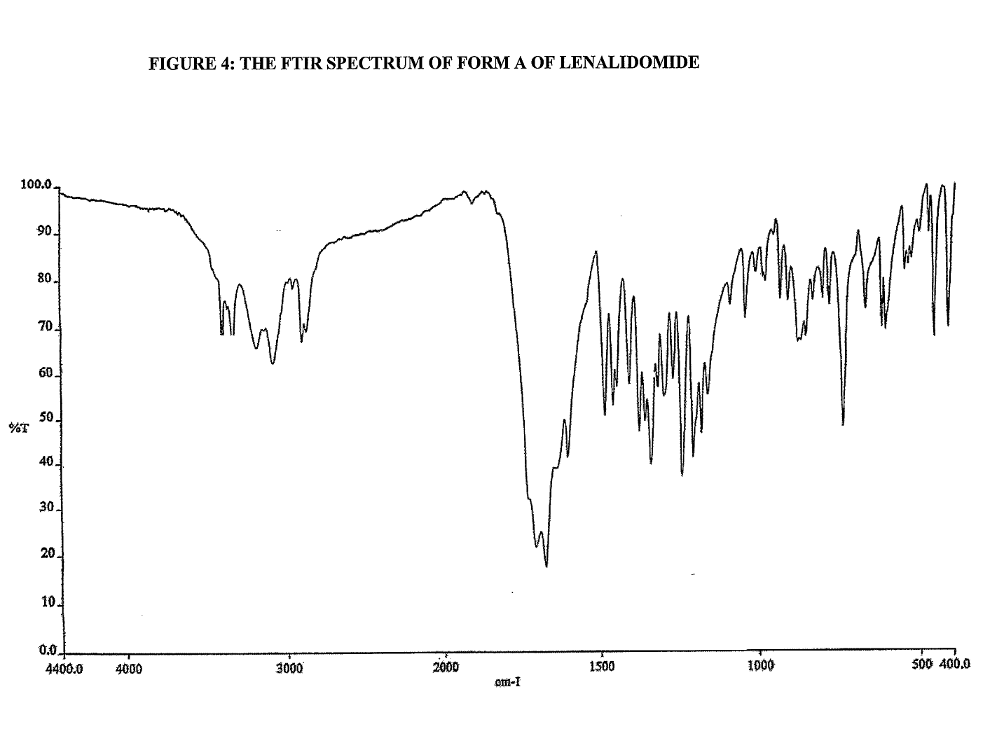

### DETAILED DESCRIPTION OF THE INVENTION

A first aspect of the present invention provides a process for the preparation of polymorphic Form A of lenalidomide, wherein the process comprises:
a) reducing 1-oxo-2-(2,6-dioxopiperidin-3-yl)-4-nitroisoindoline in N, N- dimethylformamide to obtain lenalidomide;
b) evaporating off the solvent from step a) and adding a suitable solvent to the residue; and
c) isolating polymorphic Form A of lenalidomide from the reaction mixture.

The “suitable solvent” used in step b) can be a C₁-C₅ aliphatic carboxylic ester of a C₁-C₅ aliphatic alcohol or a C₁-C₅ aliphatic alcohol optionally substituted with an alkoxy group wherein the alkoxy group contains C₁-C₅ carbon atoms. Preferably, the suitable solvent is a C₁-C₅ alkyl acetate or C₁-C₅ alkoxy ethanol. More preferably, the suitable solvent is methyl acetate or 2-methoxyethanol.

The 1-oxo-2-(2,6-dioxopiperidin-3-yl)-4-nitroisoindoline may be prepared according to the methods provided in U.S. Pat. No. 5,635,517. Reduction of 1-oxo-2- (2,6-dioxopiperidin-3-yl)-4-nitroisoindoline may be carried out using a reducing agent, for example, palladium-carbon in the presence of hydrogen atmosphere and N,N-dimethylformamide. The palladium-carbon may be about 10% to about 70% wet, for example, from about 30% to about 60% wet. The temperature of the reaction may be maintained from about 20° C. to about 60° C., for example, from about 30° C. to about 40° C. The hydrogen pressure in hydrogenator may be maintained from about 40 psi to about 70 psi. After the completion of reduction, the reaction mixture may be filtered to remove the catalyst.

The reaction mixture may be concentrated by removing N, N-dimethylformamide, for example, to obtain a solid or semisolid, prior to treatment with the suitable solvent. The treatment with methyl acetate may be carried out at a temperature from about 20° C. to about 60° C., for example, from about 40° C. to about 55° C. for about 1 hour to about 50 hours. The formation of Form A may be effected by stiffing the mixture. Form A is isolated from reaction mixture by filtration, concentration, decantation, or a combination thereof.

A second aspect of the present invention provides a process for the preparation of polymorphic Form A of lenalidomide, wherein the process comprises:
a) reducing 1-oxo-2-(2,6-dioxopiperidin-3-yl)-4-nitroisoindoline in N, N- dimethylformamide to obtain lenalidomide;
b) treating lenalidomide obtained in step a) with methyl acetate; and
c) isolating polymorphic Form A of lenalidomide from the reaction mixture thereof.

A third aspect of the present invention provides a process for the preparation of polymorphic Form A of lenalidomide, wherein the process comprises:
a) reducing 1-oxo-2-(2,6-dioxopiperidin-3-yl)-4-nitroisoindoline in N, N- dimethylformamide to obtain lenalidomide;
b) treating lenalidomide obtained in step a) with 2-methoxyethanol; and
c) isolating polymorphic Form A of lenalidomide from the reaction mixture thereof.

The 1-oxo-2-(2,6-dioxopiperidin-3-yl)-4-nitroisoindoline may be prepared according to the methods provided in U.S. Pat. No. 5,635,517. Reduction of 1-oxo-2- (2,6-dioxopiperidin-3-yl)-4-nitroisoindoline may be carried out using a reducing agent, for example, palladium-carbon in the presence of a hydrogen atmosphere and N,N-dimethylformamide.

The palladium-carbon may be about 10% to about 70% wet, for example, from about 30% to about 60% wet. The temperature of reaction may be maintained from about 20° C. to about 60° C., for example, from about 30° C. to about 40° C. for about 1 hour to about 100 hours. The hydrogen pressure in the hydrogenator may be maintained from about 40 psi to about 70 psi. After the completion of reduction, the reaction mixture may be filtered to remove the catalyst. The reaction mixture may be concentrated by removing N, N- dimethylformamide, for example, to obtain a solid or semisolid, prior to treatment with 2-methoxyethanol. The treatment with 2-methoxyethanol may be carried out by preparing a solution comprising lenalidomide in 2-methoxyethanol, for example by heating to about 70° C. to about 100° C., followed by stirring at about 15° C. to about 30° C. Form A is isolated from the reaction mixture by filtration, concentration, decantation, or a combination thereof.

XRPD of the samples were determined using X-Ray diffractometer, Rigaku Corporation, RU-H3R, Goniometer CN2155A3, X-Ray tube with Cu target anode, Power: 40 KV, 100 Ma, Scanning speed: 2 deg/min step: 0.02 deg, Wave length: 1.5406 Å.

FTIR spectra of the samples were recorded on a Perkin-Elmer 16 PC instrument, as potassium bromide pellets.

The TGA was recorded on TA (Q500) (Rate of heating=10° C./minute).

The DSC was recorded on Mettler Toledo (DSC 821) (Rate of heating=10° C./minute).

While the present invention has been described in terms of its specific embodiments, certain modifications and equivalents will be apparent to those skilled in the art and are intended to be included within the scope of the present invention.

### Example 1

### Preparation of Form A of Lenalidomide

The 1-oxo-2-(2,6-dioxopiperidin-3-yl)-4-nitroisoindoline (40 g), N, N-dimethyl formamide (500 ml) and 50% wet 10% palladium-carbon (4 g) were charged in a hydrogenator. The hydrogen pressure was maintained in the hydrogenator at 50 psi to 60 psi for 3 hours. The reaction mixture was subsequently filtered through a Celite bed and washed with N, N-dimethylformamide (100 ml). The N, N-dimethylformamide was recovered from the filtrate under vacuum at 65° C. to 70° C. to obtain a solid. Methyl acetate (200 ml) was added to the solid and the mixture was warmed to 45° C. to 50° C. The reaction mixture was stirred for 4 hours at 45° C. to 50° C., filtered, washed with methyl acetate (50 ml) and dried under vacuum at 45° C. to 50° C. to obtain the title compound.

Yield: 33.86 g

### Example 2

### Preparation of Form A of Lenalidomide

The 1-oxo-2-(2,6-dioxopiperidin-3-yl)-4-nitroisoindoline (40 g), N, N- dimethylformamide (500 ml) and 50% wet 10% palladium-carbon (4 g) were charged in the hydrogenator. The hydrogen pressure was maintained in the hydrogenator at 50 psi to 60 psi for 3 hours. The reaction mixture was subsequently filtered through a Celite bed and washed with N, N-dimethylformamide (50 ml). N, N-dimethylformamide was recovered from the filtrate under vacuum at 65° C. to 70° C. to obtain a solid. The 2-methoxyethanol (100 ml) was added to the solid and the mixture was warmed to 80° C. The reaction mixture was stirred for 3 hours at 80° C. to obtain a clear solution. The reaction mixture was cooled to 20° C. to 25° C. and stirred for 3 hours further. The mixture was filtered and dried under vacuum at 50° C. to 55° C. to obtain the title compound.

Yield: 9.0 g

We claim:

1. A process for the preparation of polymorphic Form A of lenalidomide, wherein the process comprises:
a) reducing 1-oxo-2-(2,6-dioxopiperidin-3-yl)-4-nitroisoindoline in N, N- dimethylformamide to obtain lenalidomide;
b) evaporating off the solvent from step a) and adding a suitable solvent to the residue; and
c) isolating polymorphic Form A of lenalidomide from the reaction mixture thereof.
wherein, the suitable solvent in step b) is a C₁-C₅ aliphatic carboxylic ester of a C₁-C₅ aliphatic alcohol or a C₁-C₅ aliphatic alcohol optionally substituted with an alkoxy group wherein the alkoxy group contains C₁-C₅ carbon atoms.

2. A process according to claim 1, wherein step a) further comprises concentrating the reaction mixture by removing N, N-dimethylformamide.

3. A process according to claim 1, wherein step b) is carried out at a temperature from about 20° to about 60° C.

4. A process according to claim 3, wherein step b) is carried out at a temperature from about 40° C. to about 55° C.

5. A process according to claim 1, wherein polymorphic Form A of lenalidomide is isolated from the reaction mixture by filtration, concentration, decantation; or a combination thereof.

6. A process according to claim 1, where the suitable solvent used in step b) is methyl acetate.

7. A process according to claim 1, where the suitable solvent used in step b) is 2-methoxy ethanol.

8. A process for the preparation of polymorphic Form A of lenalidomide, wherein the process comprises:
a) reducing 1-oxo-2-(2,6-dioxopiperidin-3-yl)-4-nitroisoindoline in N, N- dimethylformamide to obtain lenalidomide;
b) evaporating off the solvent from step a) and adding methyl acetate to the residue; and
c) isolating polymorphic Form A of lenalidomide from the reaction mixture thereof.

9. A process according to claim 8, wherein step a) further comprises concentrating the reaction mixture by removing N, N-dimethylformamide.

10. A process according to claim 8, wherein step b) further comprises heating the reaction mixture to about 70° C. to about 100° C. and stirring at about 15° C. to about 30° C.

11. A process according to claim 8, wherein polymorphic Form A of lenalidomide is isolated from the reaction mixture by filtration, concentration, decantation, or a combination thereof.

12. A process for the preparation of polymorphic Form A of lenalidomide, wherein the process comprises:
a) reducing 1-oxo-2-(2,6-dioxopiperidin-3-yl)-4-nitroisoindoline in N, N- dimethylformamide to obtain lenalidomide;
b) evaporating off the solvent from step a) and adding 2-methoxyethanol; and
c) isolating polymorphic Form A of lenalidomide from the reaction mixture thereof.

13. A process according to claim 12, wherein step a) further comprises concentrating the reaction mixture by removing N, N-dimethylformamide.

14. A process according to claim 12, wherein step b) further comprises heating the reaction mixture to about 70° C. to about 100° C. and stirring at about 15° C. to about 30° C.

15. A process according to claim 12, wherein polymorphic Form A of lenalidomide is isolated from the reaction mixture by filtration, concentration, decantation, or a combination thereof.
